(19) United States
(12) Patent Application Publication
(10) Pub. No.: US 2010/0247675 A1
(43) Pub. Date: Sep. 30, 2010

(54) MAGNOTHERAPY PRODUCTS

(76) Inventor: Derek Raymond PRICE, Wiltshire (GB)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/712,994

(22) Filed: Feb. 25, 2010

Related U.S. Application Data
(60) Provisional application No. 61/155,336, filed on Feb. 25, 2009.

(51) Int. Cl. A61K 33/24; A61P 35/00; A61P 15/00

(52) U.S. Cl. 424/617

(57) ABSTRACT

The present invention relates to a method of treating and/or reducing and/or preventing symptoms associated with menopause comprising applying a magnetic product to a human or animal body wherein the magnetic product comprises a magnet having positive and negative poles, the magnet having a metallic element which distorts the magnetic field distribution around the magnet so as to attenuate the magnetic field in the vicinity of the positive pole of the magnet. The invention also relates to the use of a magnetic product in the prevention and/or treatment of cancer, wherein the magnetic product comprises a magnet having positive and negative poles, the magnet having a metallic element which distorts the magnetic field distribution around the magnet so as to attenuate the magnetic field in the vicinity of the positive pole of the magnet. The invention also relates to the use of a magnetic product to increase the level of IGHBP-3 in a subject, in which the magnetic product comprises a magnet having positive and negative poles, the magnet having a metallic element which distorts the magnetic field distribution around the magnet so as to attenuate the magnetic field in the vicinity of the positive pole of the magnet.

### FIELD OF INVENTION

The present invention relates to the use of magnotherapy products, i.e. magnetic products including one or more magnets, in the treatment of menopause and/or cancer. The invention also relates to a method alleviating symptoms of menopause using magnotherapy products.

### BACKGROUND

Magnotherapy describes the practice of placing magnets adjacent an animal or human body in order to alleviate symptoms or to enhance performance of the animal or human. The mechanism by which magnotherapy works has to date not been understood or confirmed by the scientific community. A discussion of magnotherapy can be found in the book “Magnetic Health. Modern Day Healing with Magnets” by D R Price (ISBN 0953679705).

Menopause indicates the end of reproductive capacity of women and arises from the cessation of ovarian function. Menopause is a gradual process that, for most women, occurs between the ages of 47 and 55 years. It is confirmed by the absence of menstrual periods for 12 consecutive months and excludes other obvious pathologic or physiologic causes. The peri-menopause, a time of changing ovarian function, precedes the final menses by 2 to 8 years. The clinical manifestations of this transition to menopause are not well understood; however, some symptoms such as hot flashes/flushes, begin in the peri-menopause and increase in occurrence as women progress through the menopause. The specific symptoms associated with menopause vary among cultures, race/ethnicity, social groups, and persons. However, symptoms include: hot flashes, irregular periods, emotional responses, changes in sexual relationship, anxiety, feelings of doom, sudden weight gain, increased muscle tension, mood swings, marked fatigue, vaginal dryness, trouble sleeping, urinary incontinence, breast tenderness/soreness, stomach problems (e.g. bloating and/or gas), irritability, loss of libido/sex drive, inability to concentrate, painful and sore muscles and/or disturbing lapses of memory.

Hormone Replacement Therapy (HRT) is currently used to alleviate the symptoms experienced by menopausal women. Hormones used in HRT usually include one or more estrogens. Sometimes, HRT further includes one or more of a progesterone, a progestin and testosterone. However, the use of HRT is associated with risks such as venous thromboembolism and endometrial cancer (Grady et al, 1995), and it is also thought to be associated with increased risks of breast cancer (Collaborative Group on Hormonal Factors in Breast Cancer. Breast cancer and hormone replacement therapy, 1995) and ovarian cancer (Rodriguez et al, 2001).

It is understandable, given the above reported side effects, that there has been more reluctance recently on the part of women to take HRT and more caution on the part of doctors to issue it so freely.

Menopause is sometimes associated with an increased risk of cancer. IGFBP-3 is a blood marker that is currently thought to have a role in cancer prevention and induction of apoptosis (programmed ea death) in cancer cells. IGFBP-3 is thought to be a tumour suppressor. IGFBP-3 is a mediator of growth suppression signals and a putative tumour suppressor gene. The growth suppression mechanisms of IGFBP-3 have not been well clarified. IGFBP-3 mRNA is more abundantly expressed in hypoxia-related inflammatory angiogenesis and recent in vivo data suggest that IGFBP-3 has direct, IGF-independent inhibitory effects on angiogenesis. IGFBP-3, the principal carrier of IGFs in the circulation, contributes to both endocrine and autocrine/paracrine growth control; it can be induced by Growth Hormone (GH), cytokines, retinoic acid, and tumour suppressors. Induction of IGFBP-3 by the well known tumour suppressor p53 has been shown in various models that directly manipulate p53 activity.

The consensus of scientific research suggests that a reduced level of IGFBP-3 results in increased vulnerability to certain cancers including hepatocellular carcinoma, breast cancer, prostate cancer, lung cancer, melanoma, head and neck cancers. Indeed, IGFBP-3 has been reported to be down-regulated in cancer tissue.

Normal breast epithelial cells are known to exert an apoptotic effect on breast cancer cells, resulting in a potential paracrine inhibition of breast tumour development. IGFBP-3 and maspin are pro-apoptotic factors produced by normal breast epithelial cells. Immunodepletion of IGFBP-3 and maspin completely abolished the normal cell-induced apoptosis of cancer cells. Together these results indicated that normal breast epithelial cells can induce apoptosis of breast cancer cells through IGFBP-3 and maspin. These findings provide a molecular hypothesis for the long observed inhibitory effect of normal surrounding cells on breast cancer development.

In general the anti-cancer activities of IGFBP-3 are likely to be due to its tumour suppressor and apoptotic activities as mentioned above. IGFBP-3 seems to act as a tumour suppressor gene in several human cancers examined. Subramanian et al (Anticancer Res. 2007 September-October; 27(5B): 3513-8) provides evidence for a tumour suppressive function of IGFBP-3 in human breast cancer. Further evidence is provided by Li et al (Prostate. 2007 Sep. 1; 67(12): 1354-61. Hypoxia-inducible factor-1alpha (HIF-1alpha) gene polymorphisms, circulating insulin-like growth factor binding protein (IGFBP)-3 levels and prostate cancer) in relation to prostate cancer.

Although the above mentioned, and many other, studies provide evidence that IGFBP-3 is associated with cancers, to date there has been no indication of a suitable manner in which to deliver IGFBP-3 to a patient and/or to induce increased IGFBP-3 expression by a patient.

What is required is an alternative treatment of menopause. Further required is a method of delivering IGFBP-3 to a patient and/or inducing increased expression of IGFBP-3 in a patient.

### SUMMARY OF THE INVENTION

Surprisingly, the inventors have found that application of a magnetic field, preferably provided by a magnetic product such as a magnet, to a living body results in a reduction in the symptoms experienced by menopausal women.

A first aspect of the invention relates to a method of reducing symptoms of menopause by applying a magnetic field to a subject wherein the magnetic field is provided by a magnetic product comprising a magnet having positive (north seeking) and negative (south seeking) poles, together with a metallic element which distorts the magnetic field distribution around the magnet so as to accentuate the magnetic field in the vicinity of the negative pole of the magnet.

The element or directional plate may also function to reduce the magnetic field in the vicinity of the positive pole.

Symptoms of menopause may include: hot flashes, irregular periods, emotional responses, changes in sexual relationship, anxiety, feelings of doom, sudden weight gain, increased muscle tension, mood swings, marked fatigue, vaginal dryness, trouble sleeping, urinary incontinence, breast tenderness/soreness, stomach problems (bloating and gas), irritability, loss of libido/sex drive, inability to concentrate, painful and sore muscles and/or disturbing lapses of memory. It has been demonstrated that the method of the invention eliminates one or more of these symptoms.

In some embodiments of this aspect of the invention, the method of treatment increases the level of luteinising hormone and/or decreases the level of estradiol and/or increases the level of estriol and/or increases the level of IGFBP-3 in a subject wearing the magnetic product compared to a base level of any one of these hormones established in the subject prior to commencement of wearing the magnetic product.

Wearing the magnetic product can lead to changes in the level of one or more blood hormones that are consistent with a potential anti-cancer action.

Therefore, a second aspect of the invention relates to the use of a magnetic product to prevent and/or to treat cancer comprising applying a magnetic field provided by a magnetic product to a subject the magnetic product comprising a magnet having positive and negative poles, the magnet having a metallic element which distorts the magnetic field distribution around the magnet so as to accentuate the magnetic field in the vicinity of the negative pole of the magnet.

The second aspect of the invention also provides a method of preventing and/or treating cancer comprising applying a magnetic product to a human body wherein the magnetic product comprises a magnet having positive and negative poles, the magnet having a metallic element which distorts the magnetic field distribution around the magnet so as to accentuate the magnetic field in the vicinity of the negative pole of the magnet.

Notably the changes in the level of one or more blood hormones include a significant increase in blood estriol and in IGFBP-3. Therefore, the method may cause levels of estriol in the blood to increase and/or levels of IGFBP-3 to increase.

A third aspect of the invention relates to the use of a magnetic field provided by a magnetic product to increase the level of IGFBP-3 in a subject, in which the magnetic product comprises a magnet having positive and negative poles, the magnet having a metallic element which distorts the magnetic field distribution around the magnet so as to accentuate the magnetic field in the vicinity of the negative pole of the magnet.

The third aspect of the invention also relates to a method of increasing levels of IGFBP-3 in a subject, by applying a magnetic field provided by a magnetic product to the subject in which the magnetic product comprises a magnet having positive and negative poles, the magnet having a metallic element which distorts the magnetic field distribution around the magnet so as to accentuate the magnetic field in the vicinity of the negative pole of the magnet.

The subject referred to in any of the aspects of the invention may be a female, such as a menopausal female. Preferably, the subject is human.

The cancer may be a female specific cancer such as a breast cancer. Alternatively, or in addition, the cancer may be a hepatocellular carcinoma, prostate cancer, lung cancer, melanoma, head cancer or neck cancer.

The subject having cancer may be menopausal. Thus application of the magnetic product may treat menopause symptoms and cancer at the same time.

For both the first and second aspects of the invention, the ranges of magnetic strength are optimised for clinical effect while balancing the requirements of magnet size, weight and attraction of ferrous material.

Use of the magnetic product to reduce symptoms of menopause and/or to prevent or treat cancer may reduce and/or eliminate the requirement for drugs, such as HRT and/or anti-cancer drugs, and may therefore reduce the overall cost of treatment of a subject. Furthermore, there is no need to determine the level of drugs required for a given subject. The use of the magnetic product may also reduce and/or eliminate the risk of side effects associated with such drugs.

Preferably the magnetic field provided by the magnetic product is applied to a human subject, for example to the pelvic region of a female human.

In some embodiments, the magnetic product is applied to the subject's body for at least 2, 4, 6, 8, 10, 12, 14, 16, 18, 20, 22 or 24 hours per day. Preferably the magnetic product is applied to the body for 24 hours per day. Furthermore, the magnetic product may be applied to the body for from 7 to 28 days per month, for example 7 to 14 days, 7 to 21 days, 14 to 21 days, 14 to 28 days or 21 to 28 days per month. Preferably the magnetic product is applied for at least 10 days, being worn daily throughout menopause.

The magnetic product may comprise a single magnet. However, the magnetic product may comprise more than one magnet, for example 2, 3, 4, 5, or 6 magnets.

The metallic element may comprise a directional plate.

The strength of the magnet may be varied in accordance to the condition being treated. For example, the strength may be above 750 G, for example from 750 G to 5,000 G, such as from 800 G to 3,600 G as measured at the surface of the magnetic product.

In some embodiments, the magnet has a strength of about 2,000 G, for example from about 1,800 G to about 2,800 G. The magnet may comprise a directional plate to enhance and focus the magnetic field towards the user. The ranges disclosed are optimised for clinical effect while balancing the requirements of magnet size, weight and attraction of ferrous material.

In some embodiments, one or more of the magnets comprises neodymium.

### BRIEF DESCRIPTION OF THE DRAWINGS

Experimental data relating to the invention will now be described, by way of example only, with reference to the accompanying figures, FIGS. 1 to 40, in which:

FIGS. 1 to 25 are box and whisker plots relating to Appendix 1. FIGS. 1 to 23 show symptoms of menopause measured at different time points. The symptoms analysed are summarised below.

FIGS. 24 to 39 are box and whisker plots relating to Appendix 2, FIGS. 24 to 39 show hormone and other markers measured at time points. The hormones and other markers analysed are detailed below.

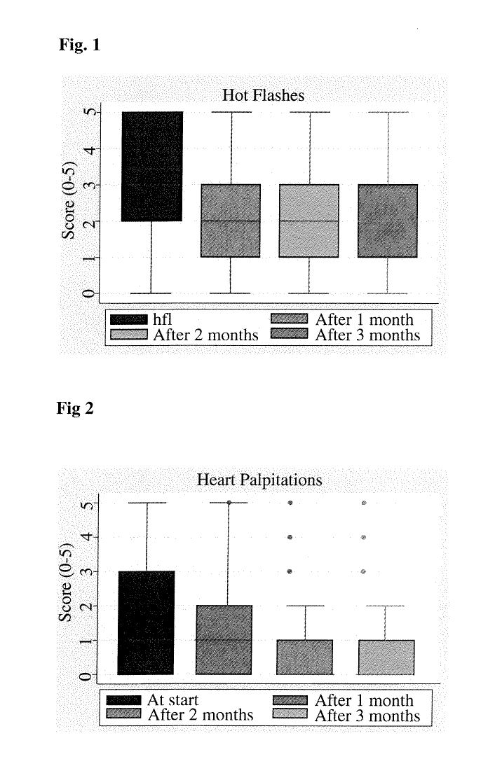

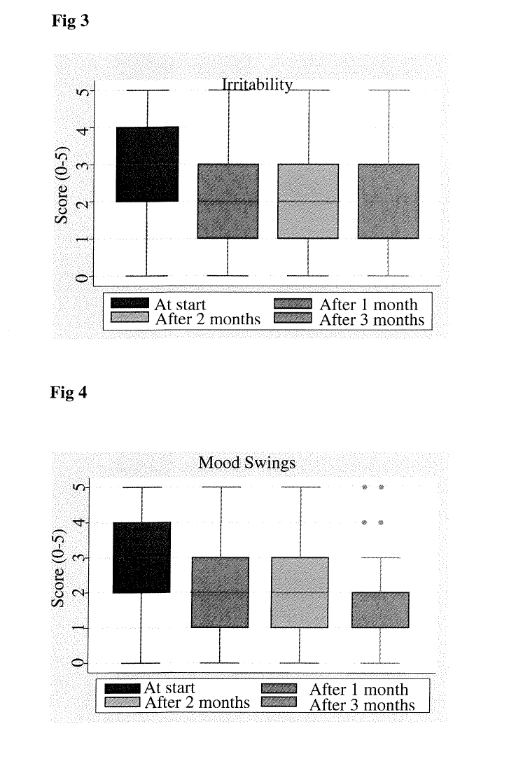

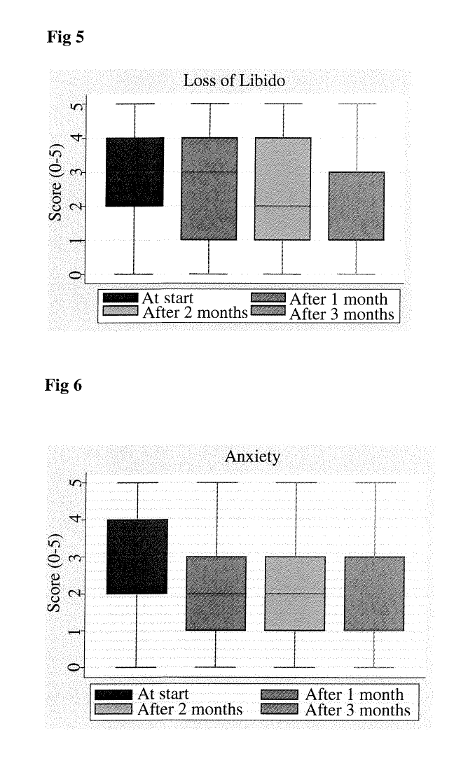

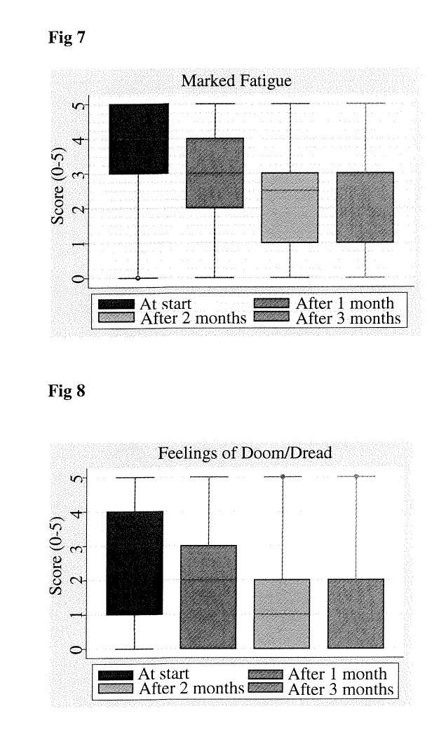

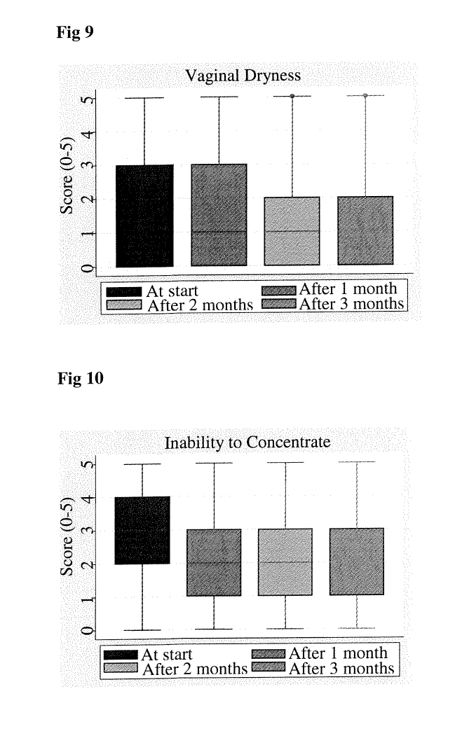

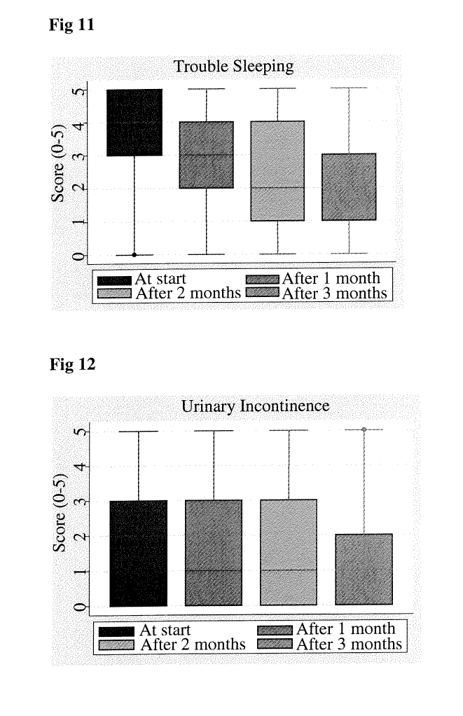

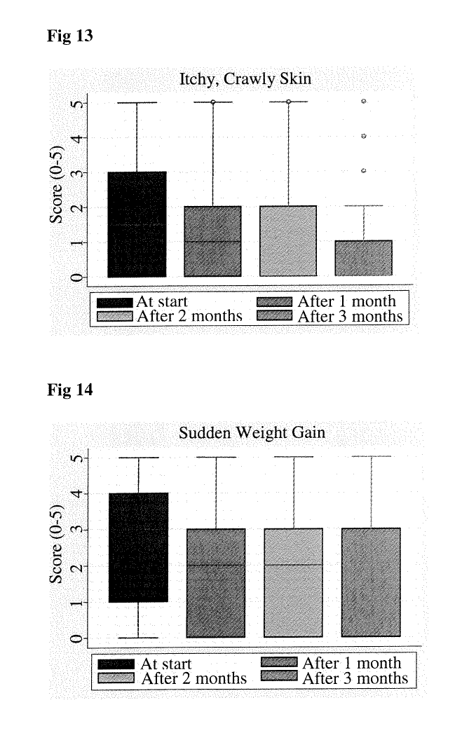

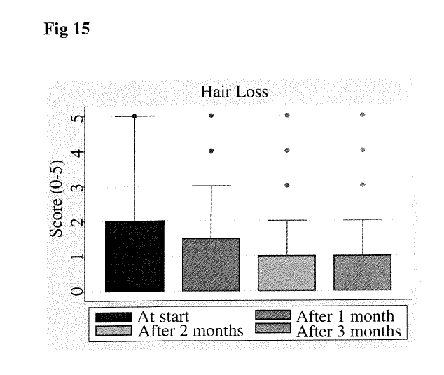

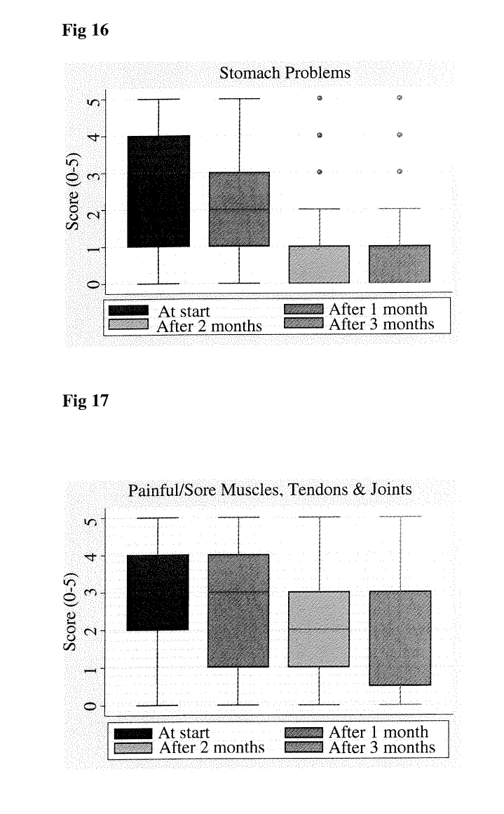

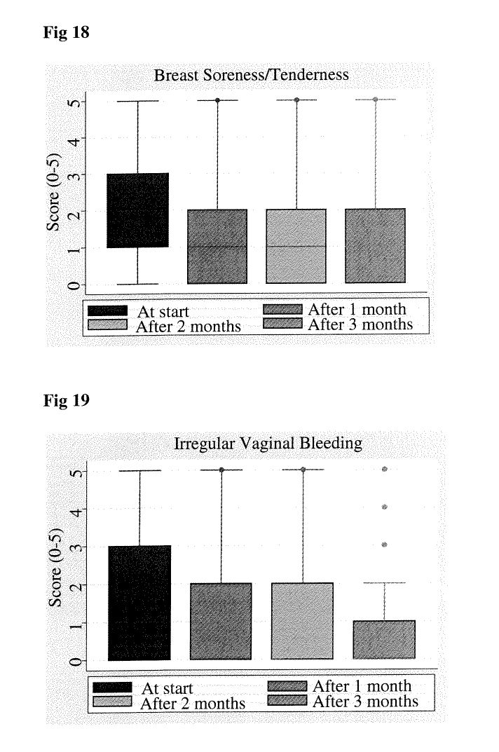

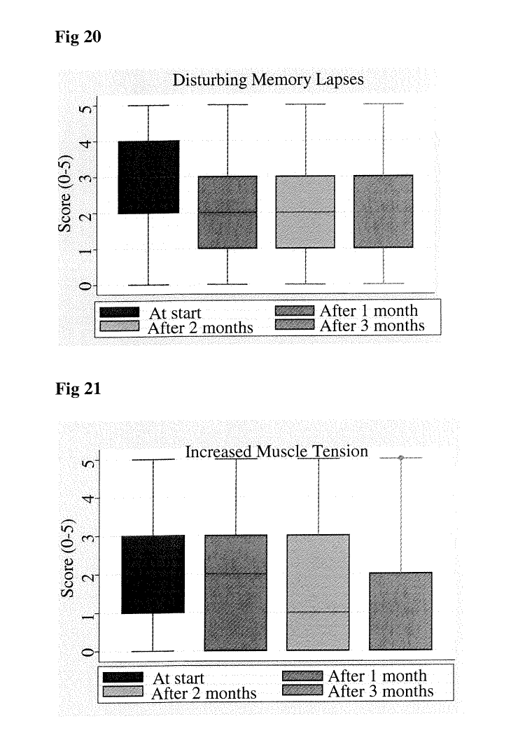

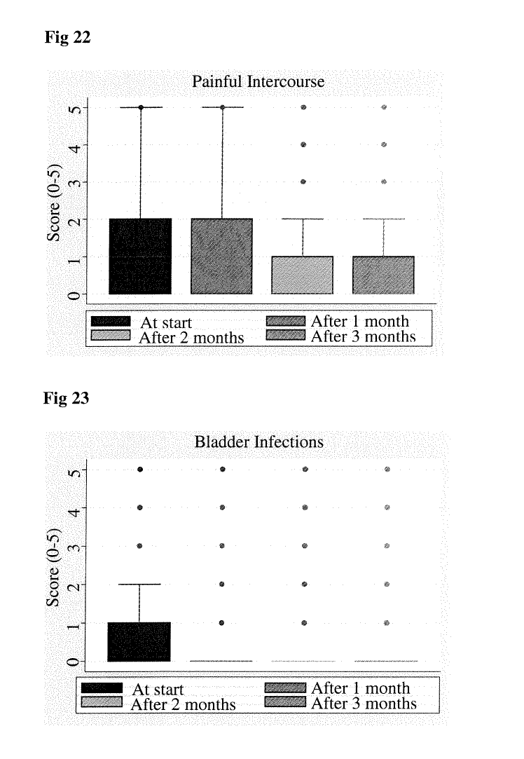

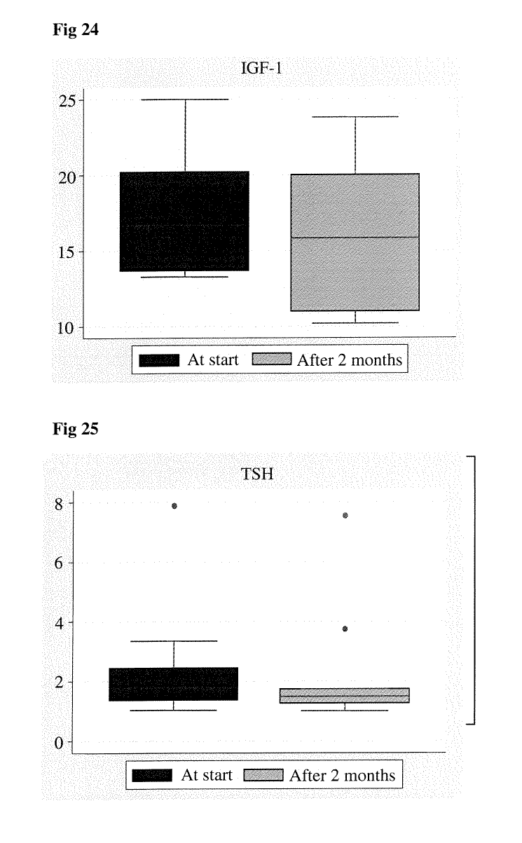

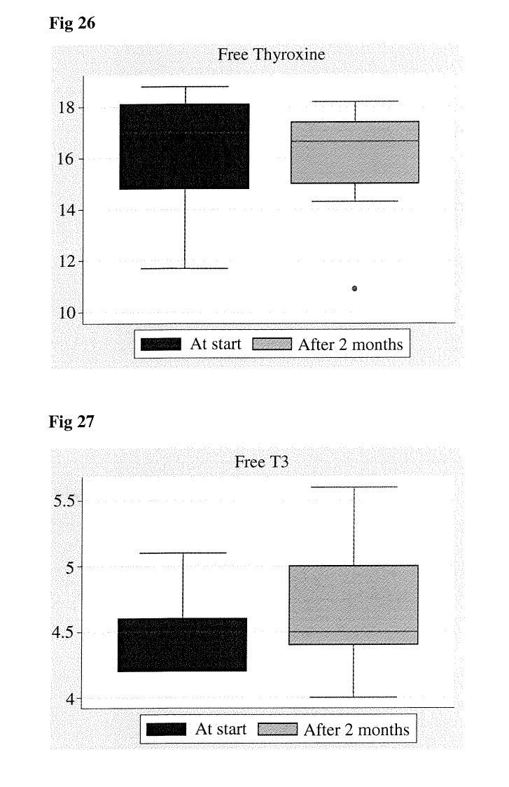

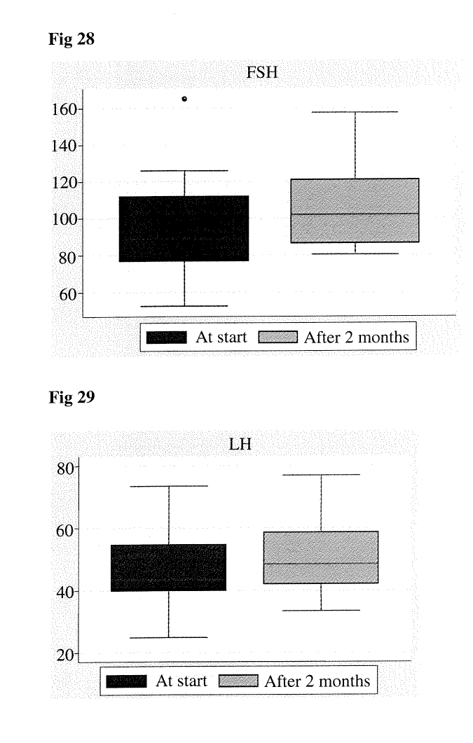

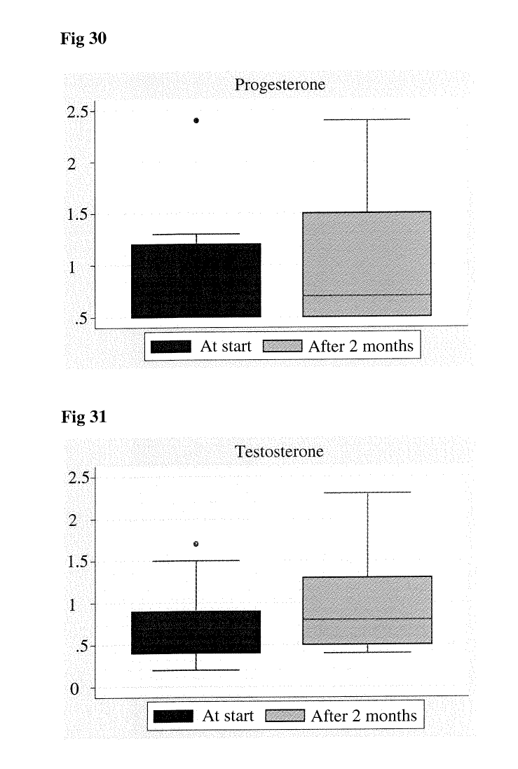

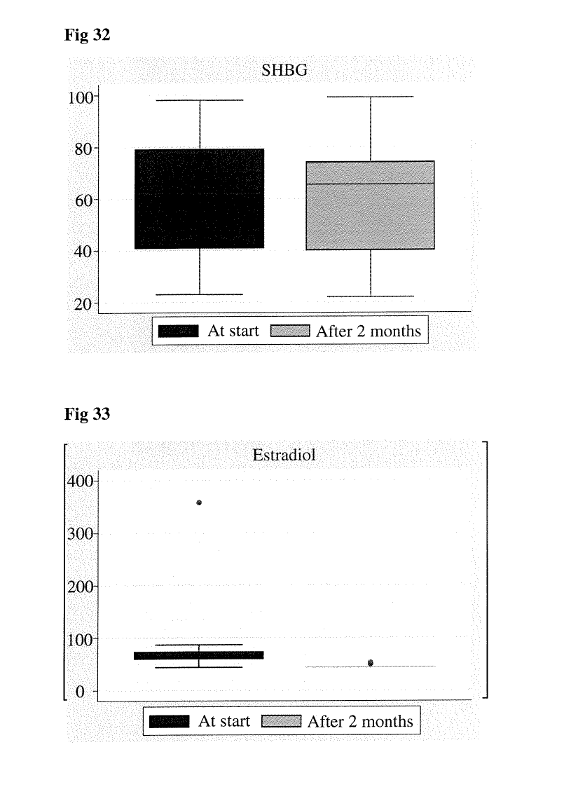

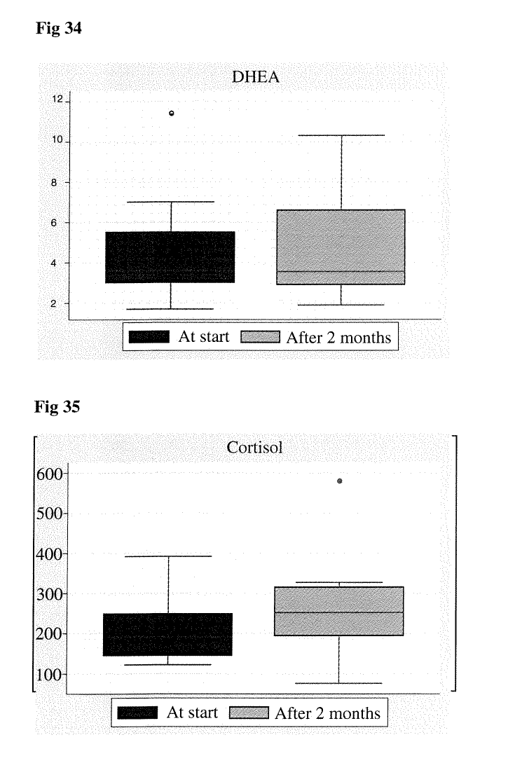

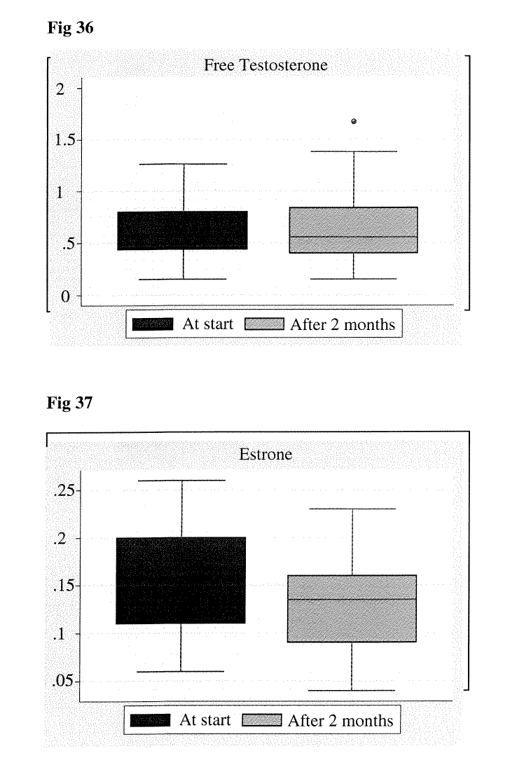

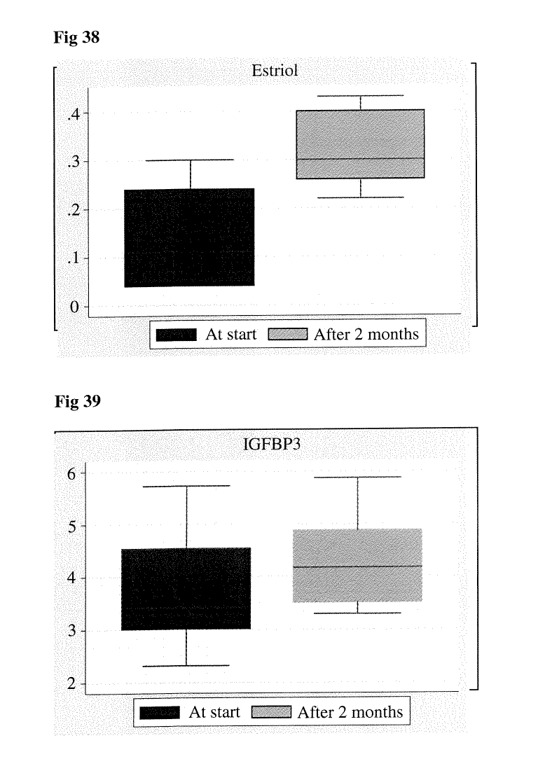

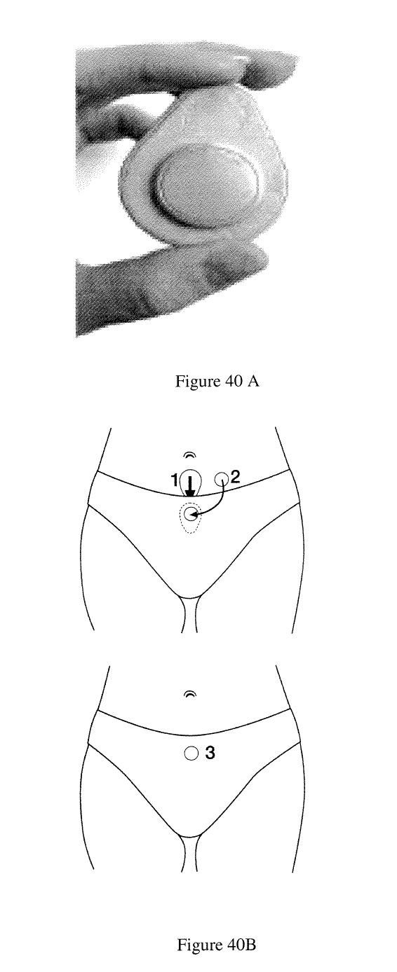

| | | | |
|---|---|---|---|
| FIG. 1: | Hot flushes | FIG. 2: | Heart palpitations |
| FIG. 3: | Irritability | FIG. 4: | Mood swings |
| FIG. 5: | Loss of libido | FIG. 6: | Anxiety |
| FIG. 7: | Marked fatigue | FIG. 8: | Feelings of doom/dread |
| FIG. 9: | Vaginal dryness | FIG. 10: | Inability to concentrate |
| FIG. 11: | Trouble sleeping | FIG. 12: | Urinary incontinence |
| FIG. 13: | Itchy, crawly skin | FIG. 14: | Sudden weight gain |
| FIG. 15: | Hair loss | FIG. 16: | Stomach problems |
| FIG. 17: | Sore muscles | FIG. 18: | Breast soreness/tenderness |
| FIG. 19: | Irregular vaginal | FIG. 20: | Lapses of memory |
| | bleeding | | |
| FIG. 21: | Muscle tension | FIG. 22: | Painful intercourse |
| FIG. 23: | Bladder infections | FIG. 24: | IGF-1 |
| FIG. 25: | TSH | FIG. 26: | Free thyroxine |
| FIG. 27: | Free T3 | FIG. 28: | FSH |
| FIG. 29: | LH | FIG. 30: | Progesterone |
| FIG. 31: | Testosterone | FIG. 32: | SHBG |
| FIG. 33: | Estradiol | FIG. 34: | DHEA |
| FIG. 35: | Cortisol | FIG. 36: | Free testosterone |
| FIG. 37: | Esterone | FIG. 38: | Estriol |
| FIG. 39: | IGFBP-3 | | |

FIG. 40 illustrates a magnetic product for use in methods and uses of the invention. FIG. 40A is a photograph of the magnetic product; FIG. 40B is a schematic diagram showing how the magnetic product is attached to a user's underwear.

In the figures, ‘S’ refers to a measurement taken at the start of the treatment, ‘1’ refers to a measurement taken one month after the start of the treatment and ‘2’ refers to a measurement taken two months after the start of the treatment.

### Experimental Data

### (a) Menopausal Symptoms (‘Descriptive Data’)

In order to analyse the effect of the magnetic device on the symptoms experienced by menopausal women, 508 menopausal women users of the magnetic device described above were surveyed. Statistically significant reductions in menopausal symptoms were reported after 1, 2 and 3 months by the women.

508 subjects were studied, having a magnetic product applied continuously except during bathing/showering. The subjects rated their menopause symptoms on a 5 point scale (where 1 is low and 5 is high) at 1, 2 and 3 months after commencing use of the magnetic device.

23 of the most common menopause symptoms were rated. The average age of the subjects was 49.7 years (±0.199). The median duration of menopause was 2.1 years (1.3-2.5 quartile ranges). It was found that use of the magnetic device caused 21 of the 23 symptoms to reduce significantly (p<10-4). The other 2 symptoms were rated as zero across the group prior to commencing use of the magnetic device, therefore no change could be determined in this study.

The following symptoms were reduced by 50 to 67%: anxiety, feelings of doom, sudden weight gain, increased muscle tension, mood swings, marked fatigue, vaginal dryness, trouble sleeping, urinary incontinence, breast tenderness/soreness. Stomach problems (bloating and gas) were reduced by 100%.

The following symptoms were reduced by 33% hot flashes, irritability, loss of libido/sex drive, inability to concentrate, painful sore muscles and disturbing lapses of memory.

Table A summarises the reported level of reduction in all the 23 symptoms in the group.

8.1% of the subjects had had a hysterectomy/oopherectomy. This did not seem to affect the response to the use of the magnetic device. There was no statistical difference in reduction of symptoms between those who still had a uterus and those that did not.

7.1% of the subjects were taking HRT. This did not seem to affect the response to the use of the magnetic device. There was no statistical difference in reduction of symptoms between those who were taking HRT and those that were not.

19.1% of the subjects lost weight through use of the magnetic device. Median weight loss was 14 pounds (8-17 pounds) over the 3 months (i.e. about 6.5 kg (about 3 to 8 kg)).

29 is of the subjects reported a return of their periods after using the magnetic device. This was usually a report of single period and not a return of regular periods. The subjects experienced one period and it was generally very light. This was experienced by subjects who were in the early stages of the menopause.

### (b) Hormones and Other Markers (‘Hormone Profile Data’)

A further study was carried out in order to analyse whether the magnetic device affected the hormone levels of the wearers. The study group consisted of 10 menopausal women (not taking HRT). The key findings are summarised in Table B. Results are shown in FIGS. 24 to 39 and the raw data is shown in like-numbered Tables 24 to 39 (Appendix 2). Note that the nomenclature d XX in these tables refers to the difference in measurement of hormones at 2 months compared to baseline (start of the trial).

Blood hormone analysis was carried out for 10 women with menopausal symptoms, but not taking HRT. The analysis was of a full blood female hormone panel (TDL labs, London) measured firstly at baseline and then repeated 2 months after daily wear of the magnetic device (applied as described below to the pelvic region, with reference to the accompanying FIG. 40). Symptoms were also measured on subjective scales exactly as they had been on the previous larger survey of 508 women. Consents for blood draw and participation in the research were obtained from all women.

The LadyCare (LC, see FIG. 40 A) magnetic device is designed for attachment to a lady's underwear by magnetic force. LC is plastic coated and comprises two parts. The first part, a pear-shaped piece, is worn inside of the lady's underwear, directly against the pelvis as shown in FIG. 40 B. The LC contains a 20-mm×5-mm neodymium iron-boron magnet within the pear-shaped piece. The second part is a circular plastic case that contains a 20-mm×1-mm neodymium iron-boron magnet with a 20-mm×2-mm 400-grade stainless steel directional plate adherent to its outside. This second part is positioned on the outside of the underwear (as shown in B) attaching securely with the force of the magnetic field. This directional plate increases the magnetic power of the north pole (standard scientific notation) of the magnetic field into the body to 2700 Gauss (surface measurement made by manufacturer at the body surface of the magnet (both parts) using a Gauss meter).

Statistically significant differences and trends to statistically significant differences in symptoms were noted in this sample of 10 women. The differences and trends identified through hormone analysis confirm the trend that had already been observed in symptom relief in the aforementioned larger survey of 508 women with menopause symptoms.

In these 10 subjects, the symptom analysis indicated trends towards significant reductions in: heart palpitations, irritability, anxiety. marked fatigue, vaginal dryness, bloating and stomach problems. Furthermore, the symptom analysis showed significant reductions in: loss of libido, trouble sleeping and night sweats.

TABLE B
Effect of wearing the magnetic device on hormone levels of menopausal
women.

| | | Effect of magnetic device |
|---|---|---|
| | Hormone | on hormone level |
| IGF1 | Insulin-like Growth Factor 1 | No change |
| TSH | Thyroid Stimulating Hormone | No change |
| — | Free Thyroxine | No change |
| Free T3 | Free Tri-iodothyronine | No change |
| FSH | Follicle Stimulating Hormone | No change |
| LH | Luteinising Hormone | Significant increase |
| | | (median 5 point |
| | | [12%] increase) |
| — | Progesterone | No change |
| — | Testosterone | No change |
| SHBG | Sex hormone binding globulin | No change |
| — | Estradiol | Significant reduction |
| | | (median 21 points |
| | | [32%] decrease) |
| DHEA | Dehydroepiandrosterone | No change |
| — | Cortisol | No change |
| — | Free testosterone | No change |
| — | Estrone | No change |
| — | Estriol | Significant increase |
| | | (median 172% increase) |
| IGFBP-3 | Insulin-like growth factor | Significant increase |
| | binding protein 3 | (median 22% increase) |

As shown in Table B, a significant reduction in the level of estradiol and significant increases in the levels of estriol and IGFBP-3 were observed.

A small (12%), but significant, increase in luteinising hormone (LH), one of the main pituitary hormones responsible for ovarian stimulation was observed. The LH level increases after menopause due to lack of negative feedback on the pituitary from reducing levels of estrogen. It is uncertain whether the small increase observed in this study may be as a result of direct stimulatory effect on the pituitary or whether it is due to the reduction in the level of estradiol (see Table B). Estradiol is one form of estrogen. The reduction in estradiol may result in a reduced level of negative feedback on the pituitary and hence an increase in the level of LH. The reduction in estrogen levels per se cannot account for the reduction in menopause symptoms.

There are three main estrogens: estrone, estradiol and estriol. It is generally thought that estriol is a less active estrogen than estradiol and that estriol seems to have protective effects in particular against hormone-related cancers, for example breast cancer. Estradiol can be converted to estriol in the body so it is possible that this conversion pathway is somehow activated by static magnetic fields, causing a rise in estriol but a fall in estradiol. Small doses of estriol have been demonstrated to remit or arrest breast cancer in menopausal women (Lemon H M et al (1975) Cancer Res 35: 1341-1352).

### (c) Comparison of Descriptive Data and Hormone Profile Data

The descriptive data (see Table A) and hormone profile data (see Table B) were analysed. As shown in Table C, there was a good correlation between the descriptive data and hormone profile data. This strongly suggests that the magnetic product is responsible for the improvement in symptoms experienced by the subjects of these studies.

TABLE C
Correlation between changes in symptoms reported by menopausal
women and changes in hormone levels measured in menopausal women
(an asterisk symbol (*) indicates a statistically significant result.)

| Hormone | Symptom | Correlation | Significance |
|---|---|---|---|
| IGF | Hot flushes | 0.6 | |
| | Urinary incontinence | 0.8 | * |
| TSH | Anxiety | −0.6 | |
| | Trouble sleeping | −0.8 | * |
| Thyroxine | Feelings of doom | 0.6 | |
| | Night sweats | −0.7 | |
| | Painful intercourse | −0.7 | |
| T3 | Anxiety | −0.7 | |
| | Itchy skin | −0.7 | |
| | Painful intercourse | −0.7 | |
| FSH | Anxiety | −0.8 | * |
| | Itchy skin | −0.6 | |
| LH | Hot flushes | 0.6 | |
| | Anxiety | −0.7 | |
| | Memory lapses | −0.6 | |
| Progesterone | Marked fatigue | −0.8 | |
| | Vaginal dryness | −0.7 | |
| | Trouble sleeping | −0.6 | |
| | Bloating | −0.7 | |
| | Weight gain | −0.8 | * |
| | Stomach problems | −0.7 | * |
| | Sore muscles | −0.7 | |
| | Breast soreness | 0.7 | |
| Testosterone | Mood swings | −0.6 | |
| | Anxiety | −0.6 | |
| | Sore muscles | −0.7 | |
| SHBG | Itchy skin | −0.6 | |
| | Stomach problems | −0.6 | |
| Estrogen | Anxiety | 0.7 | |
| | Itchy skin | 0.7 | |
| DHEA | Marked fatigue | −0.7 | |
| | Bloating | −0.8 | |
| | Stomach problems | −0.6 | |
| Cortisol | Vaginal dryness | −0.8 | |
| | Free testosterone | −0.6 | |
| | Vaginal dryness | −0.6 | |
| Estrol | Anxiety | 0.7 | |
| Estriol | Anxiety | 0.7 | |
| | Itchy skin | 0.7 | |
| IGFBP-3 | Sore muscles | −0.7 | |

As shown in Table B, use of the magnetic product induced a significant increase in the level of IGFBP-3 in the subjects. Thus the surprising finding was made that magnetic product may be useful in the prevention and/or treatment of cancers.

The data are also shown in the figures.

TABLE A

| | | | | | |
|---|---|---|---|---|---|
| | | | 25^{th} | | 75^{th} |
| N | mean | se(mean) | percentile | median | percentile |

TABLE A.1: HOT FLUSHES: (SEE ALSO FIG. 1)

| | | | | | | |
|---|---|---|---|---|---|---|
| Hot | | | | | | |
| flashes | | | | | | |
| At | 508 | 3.1 | 0.072 | 2 | 3 | 5 |
| start | | | | | | |
| After 1 | 508 | 2.3 | 0.069 | 1 | 2 | 3 |
| month | | | | | | |
| After 2 | 508 | 2.0 | 0.067 | 1 | 2 | 3 |
| months | | | | | | |
| After 3 | 508 | 1.8 | 0.065 | 1 | 2 | 3 |
| months | | | | | | |
| At | 508 | 0.9 | 0.052 | 0 | 1 | 1 |
| start - | | | | | | |
| After 1 | | | | | | |
| month* | | | | | | |
| At | 508 | 1.2 | 0.061 | 0 | 1 | 2 |
| start - | | | | | | |
| After 2 | | | | | | |
| months | | | | | | |
| At | 508 | −1.3 | 0.066 | 0 | 1 | 2 |
| start - | | | | | | |
| After 3 | | | | | | |
| months | | | | | | |

TABLE A.2: HEART PALPITATIONS: (SEE ALSO FIG. 2)

| | | | | | | |
|---|---|---|---|---|---|---|
| Heart | | | | | | |
| palpitations | | | | | | |
| At start | 508 | 1.6 | 0.064 | 0 | 1 | 3 |
| After 1 month | 508 | 1.0 | 0.052 | 0 | 1 | 2 |
| After 2 months | 508 | 0.8 | 0.045 | 0 | 0 | 1 |
| After 3 months | 508 | 0.7 | 0.043 | 0 | 0 | 1 |
| At start - | 508 | 0.6 | 0.040 | 0 | 0 | 1 |
| After 1 month* | | | | | | |
| At start - | 508 | 0.8 | 0.047 | 0 | 1 | 1 |
| After 2 months | | | | | | |
| At start - | 508 | 0.9 | 0.051 | 0 | 1 | 2 |
| After 3 months | | | | | | |

TABLE A.3: IRRITABILLITY: (SEE ALSO FIG. 3)

| | | | | | | |
|---|---|---|---|---|---|---|
| Irritability | | | | | | |
| At start | 508 | 3.2 | 0.060 | 2 | 3 | 4 |
| After 1 month | 508 | 2.4 | 0.055 | 1 | 2 | 3 |
| After 2 months | 508 | 2.0 | 0.053 | 1 | 2 | 3 |
| After 3 months | 508 | 1.8 | 0.054 | 1 | 2 | 3 |
| At start - | 508 | 0.8 | 0.042 | 0 | 1 | 1 |
| After 1 month | | | | | | |
| At start - | 508 | 1.1 | 0.049 | 0 | 1 | 2 |
| After 2 months | | | | | | |
| At start - | 508 | 1.3 | 0.053 | 0 | 1 | 2 |
| After 3 months | | | | | | |

TABLE A.4: MOOD SWINGS: (SEE ALSO FIG. 4)

| | | | | | | |
|---|---|---|---|---|---|---|
| Mood Swings | | | | | | |
| At start | 508 | 3.0 | 0.065 | 2 | 3 | 4 |
| After 1 month | 508 | 2.2 | 0.061 | 1 | 2 | 3 |
| After 2 months | 508 | 1.9 | 0.058 | 1 | 2 | 3 |
| After 3 months | 508 | 1.6 | 0.056 | 1 | 1.5 | 2 |
| At start - | 508 | 0.8 | 0.044 | 0 | 1 | 1 |
| After 1 month | | | | | | |
| At start - | 508 | 1.1 | 0.051 | 0 | 1 | 2 |
| After 2 months | | | | | | |
| At start - | 508 | 1.4 | 0.053 | 0.5 | 1 | 2 |
| After 3 months | | | | | | |

TABLE A.5: LOSS OF LIBIDO: (SEE ALSO FIG. 5)

| | | | | | | |
|---|---|---|---|---|---|---|
| Loss of Libido | | | | | | |
| At start | 508 | 3.0 | 0.075 | 2 | 3 | 4 |
| After 1 month | 508 | 2.5 | 0.073 | 1 | 3 | 4 |
| After 2 months | 508 | 2.3 | 0.072 | 1 | 2 | 4 |
| After 3 months | 508 | 2.1 | 0.071 | 1 | 2 | 3 |
| At start - | 508 | 0.5 | 0.040 | 0 | 0 | 1 |
| After 1 month | | | | | | |
| At start - | 508 | 0.7 | 0.047 | 0 | 0 | 1 |
| After 2 months | | | | | | |
| At start - | 508 | 0.9 | 0.052 | 0 | 1 | 2 |
| After 3 months | | | | | | |

TABLE A.6: ANXIETY: (SEE ALSO FIG. 6)

| | | | | | | |
|---|---|---|---|---|---|---|
| Anxiety | | | | | | |
| At start | 508 | 2.9 | 0.068 | 2 | 3 | 4 |
| After 1 month | 508 | 2.2 | 0.064 | 1 | 2 | 3 |
| After 2 months | 508 | 1.9 | 0.060 | 1 | 2 | 3 |
| After 3 months | 508 | 1.7 | 0.060 | 1 | 1 | 3 |
| At start - | 508 | 0.7 | 0.042 | 0 | 1 | 1 |
| After 1 month | | | | | | |
| At start - | 508 | 1.0 | 0.046 | 0 | 1 | 2 |
| After 2 months | | | | | | |
| At start - | 508 | 1.2 | 0.052 | 0 | 1 | 2 |
| After 3 months | | | | | | |

TABLE A.7: MARKED FATIGUE: (SEE ALSO, FIG. 7)

| | | | | | | |
|---|---|---|---|---|---|---|
| Marked fatigue | | | | | | |
| At start | 508 | 3.5 | 0.057 | 3 | 4 | 5 |
| After 1 month | 508 | 2.8 | 0.059 | 2 | 3 | 4 |
| After 2 months | 508 | 2.5 | 0.061 | 1 | 2.5 | 3 |
| After 3 months | 508 | 2.2 | 0.061 | 1 | 2 | 3 |
| At start - | 508 | 0.7 | 0.042 | 0 | 1 | 1 |
| After 1 month | | | | | | |
| At start - | 508 | 1.0 | 0.051 | 0 | 1 | 2 |
| After 2 months | | | | | | |
| At start - | 508 | 1.3 | 0.056 | 0 | 1 | 2 |
| After 3 months | | | | | | |

TABLE A.8: FEELINGS OF DOOM/DREAD (SEE ALSO, FIG. 8)

| | | | | | | |
|---|---|---|---|---|---|---|
| Feelings of | | | | | | |
| doom/dread | | | | | | |
| At start | 508 | 2.4 | 0.076 | 1 | 3 | 4 |
| After 1 month | 508 | 1.8 | 0.067 | 0 | 2 | 3 |
| After 2 months | 508 | 1.4 | 0.061 | 0 | 1 | 2 |
| After 3 months | 508 | 1.3 | 0.059 | 0 | 1 | 2 |
| At start - | 508 | 0.7 | 0.042 | 0 | 0 | 1 |
| After 1 month | | | | | | |
| At start - | 508 | 1.0 | 0.053 | 0 | 1 | 2 |
| After 2 months | | | | | | |
| At start - | 508 | 1.1 | 0.057 | 0 | 1 | 2 |
| After 3 months | | | | | | |

TABLE A.9: VAGINAL DRYNESS: (SEE ALSO, FIG. 9)

| | | | | | | |
|---|---|---|---|---|---|---|
| Vaginal Dryness | | | | | | |
| At start | 508 | 2.0 | 0.073 | 0 | 2 | 3 |
| After 1 month | 508 | 1.7 | 0.068 | 0 | 1 | 3 |
| After 2 months | 508 | 1.5 | 0.063 | 0 | 1 | 2 |
| After 3 months | 508 | 1.3 | 0.060 | 0 | 1 | 2 |
| At start - | 508 | 0.3 | 0.030 | 0 | 0 | 1 |
| After 1 month | | | | | | |
| At start - | 508 | 0.6 | 0.040 | 0 | 0 | 1 |
| After 2 months | | | | | | |
| At start - | 508 | 0.7 | 0.046 | 0 | 0 | 1 |
| After 3 months | | | | | | |

TABLE A.10: INABILITY TO CONCENTRATE: (SEE ALSO, FIG. 10)

| | | | | | | |
|---|---|---|---|---|---|---|
| Inability to | | | | | | |
| Concentrate | | | | | | |
| At start | 508 | 2.8 | 0.608 | 2 | 3 | 4 |
| After 1 month | 508 | 2.3 | 0.059 | 1 | 2 | 3 |
| After 2 months | 508 | 2.0 | 0.056 | 1 | 2 | 3 |
| After 3 months | 508 | 1.8 | 0.055 | 1 | 2 | 3 |
| At start - | 508 | 0.5 | 0.036 | 0 | 0 | 1 |
| After 1 month | | | | | | |
| At start - | 508 | 0.8 | 0.044 | 0 | 1 | 1 |
| After 2 months | | | | | | |
| At start - | 508 | 1.0 | 0.050 | 0 | 1 | 2 |
| After 3 months | | | | | | |

TABLE A.11: TROUBLE SLEEPING: (SEE ALSO, FIG. 11)

| | | | | | | |
|---|---|---|---|---|---|---|
| Trouble | | | | | | |
| Sleeping | | | | | | |
| At start | 508 | 3.7 | 0.061 | 3 | 4 | 5 |
| After 1 month | 508 | 2.8 | 0.066 | 2 | 3 | 4 |
| After 2 months | 508 | 2.4 | 0.067 | 1 | 2 | 4 |
| After 3 months | 508 | 2.2 | 0.067 | 1 | 2 | 3 |
| At start - | 508 | 0.9 | 0.047 | 0 | 1 | 1 |
| After 1 month | | | | | | |
| At start - | 508 | 1.2 | 0.056 | 0 | 1 | 2 |
| After 2 months | | | | | | |
| At start - | 508 | 1.5 | 0.063 | 1 | 1 | 2 |
| After 3 months | | | | | | |

TABLE A.12: URINARY INCONTINENCE: (SEE ALSO FIG. 12)

| | | | | | | |
|---|---|---|---|---|---|---|
| Urinary | | | | | | |
| Incontinence | | | | | | |
| At start | 508 | 2.0 | 0.077 | 0 | 2 | 3 |
| After 1 month | 508 | 1.7 | 0.071 | 0 | 1 | 3 |
| After 2 months | 508 | 1.5 | 0.068 | 0 | 1 | 3 |
| After 3 months | 508 | 1.3 | 0.064 | 0 | 1 | 2 |
| At start - | 508 | 0.4 | 0.036 | 0 | 0 | 1 |
| After 1 month | | | | | | |
| At start - | 508 | 0.6 | 0.046 | 0 | 0 | 1 |
| After 2 months | | | | | | |
| At start - | 508 | 0.7 | 0.050 | 0 | 0 | 1 |
| After 3 months | | | | | | |

TABLE A.13: ITCHY, CRAWLY SKIN: (SEE ALSO FIG. 13)

| | | | | | | |
|---|---|---|---|---|---|---|
| Itchy, Crawly | | | | | | |
| Skin | | | | | | |
| At start | 508 | 1.7 | 0.073 | 0 | 1.5 | 3 |
| After 1 month | 508 | 1.2 | 0.063 | 0 | 1 | 2 |
| After 2 months | 508 | 1.0 | 0.058 | 0 | 0 | 2 |
| After 3 months | 508 | 0.8 | 0.052 | 0 | 0 | 1 |
| At start - | 508 | 0.5 | 0.041 | 0 | 0 | 1 |
| After 1 month | | | | | | |
| At start - | 508 | 0.7 | 0.053 | 0 | 0 | 1 |
| After 2 months | | | | | | |
| At start - | 508 | 0.9 | 0.059 | 0 | 0 | 2 |
| After 3 months | | | | | | |

TABLE A.14: SUDDEN WEIGHT GAIN: (SEE ALSO FIG. 14)

| | | | | | | |
|---|---|---|---|---|---|---|
| Sudden Weight | | | | | | |
| Gain | | | | | | |
| At start | 508 | 2.4 | 0.077 | 1 | 3 | 4 |
| After 1 month | 508 | 2.1 | 0.076 | 0 | 2 | 3 |
| After 2 months | 508 | 1.8 | 0.074 | 0 | 2 | 3 |
| After 3 months | 508 | 1.6 | 0.073 | 0 | 1 | 3 |
| At start - | 508 | 0.3 | 0.043 | 0 | 0 | 1 |
| After 1 month | | | | | | |
| At start - | 508 | 0.6 | 0.052 | 0 | 0 | 1 |
| After 2 months | | | | | | |
| At start - | 508 | 0.8 | 0.058 | 0 | 0.5 | 1 |
| After 3 months | | | | | | |

TABLE A.15: HAIR LOSS: (SEE ALSO FIG. 15)

| | | | | | | |
|---|---|---|---|---|---|---|
| Hair Loss | | | | | | |
| At start | 508 | 1.0 | 0.064 | 0 | 0 | 2 |
| After 1 | 508 | 0.9 | 0.061 | 0 | 0 | 1.5 |
| month | | | | | | |
| After 2 | 508 | 0.8 | 0.055 | 0 | 0 | 1 |
| months | | | | | | |
| After 3 | 508 | 0.7 | 0.053 | 0 | 0 | 1 |
| months | | | | | | |
| At start - | 508 | 0.1 | 0.028 | 0 | 0 | 0 |
| After 1 | | | | | | |
| month | | | | | | |
| At start - | 508 | 0.2 | 0.034 | 0 | 0 | 0 |
| After 2 | | | | | | |
| months | | | | | | |
| At start - | 508 | 0.3 | 0.040 | 0 | 0 | 1 |
| After 3 | | | | | | |
| months | | | | | | |

TABLE A.16: STOMACH PROBLEMS: (SEE ALSO FIG. 16)

| | | | | | | |
|---|---|---|---|---|---|---|
| Stomach | | | | | | |
| Problems | | | | | | |
| At start | 508 | 2.6 | 0.073 | 1 | 3 | 4 |
| After 1 month | 508 | 2.1 | 0.067 | 1 | 2 | 3 |
| After 2 months | 508 | 0.9 | 0.054 | 0 | 0 | 1 |
| After 3 months | 508 | 0.9 | 0.051 | 0 | 0 | 1 |
| At start - | 508 | 0.5 | 0.039 | 0 | 0 | 1 |
| After 1 month | | | | | | |
| At start - | 508 | 1.8 | 0.077 | 0 | 2 | 3 |
| After 2 months | | | | | | |
| At start - | 508 | 1.8 | 0.069 | 0 | 2 | 3 |
| After 3 months | | | | | | |

TABLE A.17: SORE MUSCLES: (SEE ALSO FIG. 17)

| | | | | | | |
|---|---|---|---|---|---|---|
| Painful/Sore | | | | | | |
| Muscles, | | | | | | |
| Tendons, Joints | | | | | | |
| At start | 508 | 2.9 | 0.073 | 2 | 3 | 4 |
| After 1 month | 508 | 2.5 | 0.069 | 1 | 3 | 4 |
| After 2 months | 508 | 2.1 | 0.069 | 1 | 2 | 3 |
| After 3 months | 508 | 1.9 | 0.068 | 0.5 | 2 | 3 |
| At start - | 508 | 0.4 | 0.040 | 0 | 0 | 1 |
| After 1 month | | | | | | |
| At start - | 508 | 0.8 | 0.054 | 0 | 1 | 1 |
| After 2 months | | | | | | |
| At start - | 508 | 1.0 | 0.059 | 0 | 1 | 2 |
| After 3 months | | | | | | |

TABLE A.18: BREAST SORENESS/TENDERNESS: (SEE ALSO FIG. 18)

| | | | | | | |
|---|---|---|---|---|---|---|
| Breast | | | | | | |
| Soreness/ | | | | | | |
| Tenderness | | | | | | |
| At start | 508 | 2.0 | 0.071 | 1 | 2 | 3 |
| After 1 month | 508 | 1.4 | 0.060 | 0 | 1 | 2 |
| After 2 months | 508 | 1.2 | 0.055 | 0 | 1 | 2 |
| After 3 months | 508 | 1.0 | 0.053 | 0 | 1 | 2 |
| At start - | 508 | 0.6 | 0.046 | 0 | 0 | 1 |
| After 1 month | | | | | | |
| At start - | 508 | 0.8 | 0.055 | 0 | 1 | 1 |
| After 2 months | | | | | | |
| At start - | 508 | 1.0 | 0.062 | 0 | 1 | 2 |
| After 3 months | | | | | | |

TABLE A.19: IRREGULAR VAGINAL BLEEDING: (SEE ALSO FIG. 19)

| | | | | | | |
|---|---|---|---|---|---|---|
| Irregular | | | | | | |
| vaginal | | | | | | |
| Bleeding | | | | | | |
| At start | 508 | 1.8 | 0.079 | 0 | 1 | 3 |
| After 1 month | 508 | 1.2 | 0.070 | 0 | 0 | 2 |
| After 2 months | 508 | 0.9 | 0.058 | 0 | 0 | 2 |
| After 3 months | 508 | 0.7 | 0.055 | 0 | 0 | 1 |
| At start - | 508 | 0.5 | 0.052 | 0 | 0 | 1 |
| After 1 month | | | | | | |
| At start - | 508 | 0.9 | 0.062 | 0 | 0 | 2 |
| After 2 months | | | | | | |
| At start - | 508 | 1.0 | 0.067 | 0 | 0 | 2 |
| After 3 months | | | | | | |

TABLE A.20: LAPSES OF MEMORY: (SEE ALSO FIG. 20)

| | | | | | | |
|---|---|---|---|---|---|---|
| Disturbing | | | | | | |
| Memory Lapses | | | | | | |
| At start | 508 | 2.7 | 0.064 | 2 | 3 | 4 |
| After 1 month | 508 | 2.3 | 0.062 | 1 | 2 | 3 |
| After 2 months | 508 | 2.0 | 0.060 | 1 | 2 | 3 |
| After 3 months | 508 | 1.8 | 0.059 | 1 | 2 | 3 |
| At start - | 508 | 0.4 | 0.037 | 0 | 0 | 1 |
| After 1 month | | | | | | |
| At start - | 508 | 0.7 | 0.047 | 0 | 1 | 1 |
| After 2 months | | | | | | |
| At start - | 508 | 1.0 | 0.051 | 0 | 1 | 2 |
| After 3 months | | | | | | |

TABLE A.21: MUSCLE TENSION: (SEE ALSO FIG. 21)

| | | | | | | |
|---|---|---|---|---|---|---|
| Increased | | | | | | |
| Muscle Tension | | | | | | |
| At start | 508 | 2.3 | 0.069 | 1 | 3 | 3 |
| After 1 month | 508 | 1.9 | 0.064 | 0 | 2 | 3 |
| After 2 months | 508 | 1.6 | 0.060 | 0 | 1 | 3 |
| After 3 months | 508 | 1.4 | 0.058 | 0 | 1 | 2 |
| At start - | 508 | 0.5 | 0.036 | 0 | 0 | 1 |
| After 1 month | | | | | | |
| At start - | 508 | 0.7 | 0.048 | 0 | 0 | 1 |
| After 2 months | | | | | | |
| At start - | 508 | 0.9 | 0.055 | 0 | 1 | 2 |
| After 3 months | | | | | | |

TABLE A.22: PAINFUL INTERCOURSE: (SEE ALSO FIG. 22)

| | | | | | | |
|---|---|---|---|---|---|---|
| Painful | | | | | | |
| Intercourse | | | | | | |
| At start | 508 | 1.3 | 0.069 | 0 | 1 | 2 |
| After 1 month | 508 | 1.0 | 0.063 | 0 | 0 | 2 |
| After 2 months | 508 | 0.9 | 0.058 | 0 | 0 | 1 |
| After 3 months | 508 | 0.8 | 0.054 | 0 | 0 | 1 |
| At start - | 508 | 0.3 | 0.029 | 0 | 0 | 0 |
| After 1 month | | | | | | |
| At start - | 508 | 0.4 | 0.039 | 0 | 0 | 1 |
| After 2 months | | | | | | |
| At start - | 508 | 0.6 | 0.043 | 0 | 0 | 1 |
| After 3 months | | | | | | |

TABLE A.23: BLADDER INFECTIONS: (SEE ALSO FIG. 23)

| | | | | | | |
|---|---|---|---|---|---|---|
| Bladder | | | | | | |
| Infections | | | | | | |
| At start | 508 | 0.7 | 0.055 | 0 | 0 | 1 |
| After 1 month | 508 | 0.4 | 0.044 | 0 | 0 | 0 |
| After 2 months | 508 | 0.3 | 0.035 | 0 | 0 | 0 |
| After 3 months | 508 | 0.2 | 0.027 | 0 | 0 | 0 |
| At start - | 508 | 0.3 | 0.036 | 0 | 0 | 0 |
| After 1 month | | | | | | |
| At start - | 508 | 0.4 | 0.043 | 0 | 0 | 1 |
| After 2 months | | | | | | |
| At start - | 508 | 0.5 | 0.048 | 0 | 0 | 1 |
| After 3 months | | | | | | |

Appendix 2. HORMONE LEVELS

| | | | | |
|---|---|---|---|---|
| | Standard error | | | |
| Mean | (mean) | p25 | p50 | p75 |

TABLE A.24: IGF-1 (SEE ALSO FIG. 24)

| | | | | | | |
|---|---|---|---|---|---|---|
| igf1 | At start | 17.49 | 1.294729 | 13.7 | 16.7 | 20.2 |
| igf2 | After 2 | 16.36 | 1.552002 | 11 | 15.85 | 20 |
| | months | | | | | |
| d_igf | 2-0 | 1.13 | 0.9262889 | −0.5 | 1.45 | 2.7 |

TABLE A.25: TSH (SEE ALSO FIG. 25)

| | | | | | | |
|---|---|---|---|---|---|---|
| tsh | At start | 2.451 | 0.6367495 | 1.37 | 1.785 | 2.44 |
| tsh2 | After 2 | 2.232 | 0.6354435 | 1.26 | 1.49 | 1.74 |
| | months | | | | | |
| d_tsh | 2-0 | 0.219 | 0.1235084 | −0.04 | 0.165 | 0.35 |

TABLE A.26: FREE THYROXINE (SEE ALSO FIG. 26)

| | | | | | | |
|---|---|---|---|---|---|---|
| thyr1 | At start | 16.24 | 0.7248294 | 14.8 | 17 | 18.1 |
| thyr2 | After 2 | 16 | 0.6764286 | 15 | 16.65 | 17.4 |
| | months | | | | | |
| d_thyr | 2-0 | 0.2399997 | 0.284097 | −0.2000008 | −0.0500007 | 1.1 |

TABLE A.27: FREE T3 (SEE ALSO FIG. 27)

| | | | | | | |
|---|---|---|---|---|---|---|
| t31 | At start | 4.29 | 0.1754043 | 4.2 | 4.5 | 4.6 |
| t32 | After 2 | 4.64 | 0.1439135 | 4.4 | 4.5 | 5 |
| | months | | | | | |
| d_t3 | 2-0 | −0.3500001 | 0.1424001 | −0.5999999 | −0.3500001 | 0.0999999 |

TABLE A.28: FSH (SEE ALSO FIG. 28)

| | | | | | | |
|---|---|---|---|---|---|---|
| fsh1 | At start | 96.31 | 9.96656 | 76.8 | 88.95 | 111.9 |
| fsh2 | After 2 | 108.13 | 7.85246 | 86.4 | 101.85 | 120.9 |
| | months | | | | | |
| d_fsh | 2-0 | −11.82 | 6.513419 | −12.6 | −10.4 | 2.100006 |

TABLE A.29: LH (SEE ALSO FIG. 29) (STATISTICALLY SIGNIFICANT
CHANGE)

| | | | | | | |
|---|---|---|---|---|---|---|
| 1h1 | At start | 46.56 | 4.179187 | 40 | 43.45 | 54.7 |
| 1h2 | After 2 | 51.26 | 4.059042 | 42 | 48.45 | 58.6 |
| | months | | | | | |
| d_1h | 2-0 | −4.699999 | 2.162355 | −4.599998 | −2.950001 | −2 |

TABLE A.30: PROGESTERONE (SEE ALSO FIG. 30)

| | | | | | | |
|---|---|---|---|---|---|---|
| prog1 | At start | 1 | 0.1849925 | 0.5 | 0.9 | 1.2 |
| prog2 | After 2 | 1.02 | 0.2009975 | 0.5 | 0.7 | 1.5 |
| | months | | | | | |
| d_prog1 | 2-0 | −0.02 | 0.0553775 | 0 | 0 | 0 |

TABLE A.31: TESTOSTERONE (SEE ALSO FIG. 31)

| | | | | | | |
|---|---|---|---|---|---|---|
| test1 | At start | 0.77 | 0.1591994 | 0.4 | 0.7 | 0.9 |
| test2 | After 2 | 0.98 | 0.1884439 | 0.5 | 0.8 | 1.3 |
| | months | | | | | |
| d_test | 2-0 | −0.21 | 0.1015983 | −.5999999 | −0.1 | −0.1 |

TABLE A.32: SHBG (SEE ALSO FIG. 32)

| | | | | | | |
|---|---|---|---|---|---|---|
| shbg1 | At start | 60.1 | 7.469122 | 41 | 62 | 79 |
| shbg2 | After 2 | 61.2 | 8.41797 | 40 | 65.5 | 74 |
| | months | | | | | |
| d_shbg | 2-0 | −1.1 | 3.634556 | −1 | 0.5 | 5 |

TABLE A.33: ESTRADIOL (SEE ALSO FIG. 33) (STATISTICALLY
SIGNIFICANT CHANGE)

| | | | | | | |
|---|---|---|---|---|---|---|
| estr1 | At start | 93.1 | 29.57982 | 60 | 65 | 73 |
| estr2 | After 2 | 45.4 | 0.9451631 | 44 | 44 | 44 |
| | months | | | | | |
| d_estr | 2-0 | 47.7 | 29.75046 | 10 | 18 | 29 |

TABLE A.34: DHEA (SEE ALSO FIG. 34)

| | | | | | | |
|---|---|---|---|---|---|---|
| dhea1 | At start | 4.66 | 0.9045809 | 3 | 3.6 | 5.5 |
| dhea2 | After 2 | 4.56 | 0.8523432 | 2.9 | 3.55 | 6.6 |
| | months | | | | | |
| d_dhea | 2-0 | 0.1 | 0.246306 | −0.1999999 | 0.3 | 0.5000001 |

TABLE A.35: CORTISOL (SEE ALSO FIG. 35)

| | | | | | | |
|---|---|---|---|---|---|---|
| cort1 | At start | 209.7 | 25.21157 | 146 | 192.5 | 249 |
| cort2 | After 2 | 262.9 | 44.40557 | 195 | 252 | 315 |
| | months | | | | | |
| d_cort | 2-0 | −53.2 | 28.26415 | −123 | −65 | 17 |

TABLE A.36: FREE TESTOSTERONE (SEE ALSO FIG. 36)

| | | | | | | |
|---|---|---|---|---|---|---|
| ftesto1 | At start | 0.579 | 0.0980867 | 0.44 | 0.47 | 0.8 |
| ftesto2 | After 2 | 0.697 | 0.1579877 | 0.4 | 0.555 | 0.84 |
| | months | | | | | |
| d_ftesto | 2-0 | −0.118 | 0.099463 | −0.22 | −0.035 | 0.06 |

TABLE A.37: ESTRONE (SEE ALSO FIG. 37)

| | | | | | | |
|---|---|---|---|---|---|---|
| estro1 | At start | 0.162 | 0.0198214 | 0.11 | 0.15 | 0.2 |
| estro2 | After 2 | 0.135 | 0.0174642 | 0.09 | 0.135 | 0.16 |
| | months | | | | | |
| d_estro | 2-0 | 0.027 | 0.0174515 | 0 | 0.02 | 0.03 |

TABLE A.38: ESTRIOL (SEE ALSO FIG. 38) STATISTICALLY
SIGNIFICANT CHANGE

| | | | | | | |
|---|---|---|---|---|---|---|
| estri1 | At start | 0.143 | 0.0359026 | 0.04 | 0.11 | 0.24 |
| estri2 | After 2 | 0.319 | 0.0236385 | 0.26 | 0.3 | 0.4 |
| | months | | | | | |
| d_estri | 2-0 | −0.176 | 0.0485157 | −0.29 | −0.205 | −0.07 |

TABLE A.39: IGFBP-3 (SEE ALSO FIG. 39) STATISTICALLY
SIGNIFICANT CHANGE

| | | | | | | |
|---|---|---|---|---|---|---|
| igfbp1 | At start | 3.782 | 0.3444796 | 3.01 | 3.415 | 4.54 |
| igfbp2 | After 2 | 4.237 | 0.2565153 | 3.51 | 4.175 | 4.88 |
| | months | | | | | |
| d_igfbp | 2-0 | −0.4550001 | 0.1650404 | −1.01 | −0.3100002 | −0.1500001 |

1. A method of treating and/or reducing and/or preventing symptoms associated with menopause comprising applying a magnetic product to a human or animal body wherein the magnetic product comprises a magnet having positive and negative poles, the magnet having a metallic element which distorts the magnetic field distribution around the magnet so as to accentuate the magnetic field in the vicinity of the negative pole of the magnet.

2. A method according to claim 1 wherein the symptoms of menopause include one or more of: hot flashes, irregular periods, emotional responses, changes in sexual relationship, anxiety, feelings of doom, sudden weight gain, increased muscle tension, mood swings, marked fatigue, vaginal dryness, trouble sleeping, urinary incontinence, breast tenderness/soreness, stomach problems (e-g. bloating and gas), irritability, loss of libido/sex drive, inability to concentrate, painful and sore muscles and/or disturbing lapses of memory.

3. (canceled)

4. A method according to any one of claims 1 or 2 wherein the method increases the level of luteinising hormone and/or decreases the level of estradiol and/or increases the level of estriol and/or increases the level of IGFBP-3 in a subject wearing the magnetic product compared to a base level established in the subject prior to application of the magnetic product and/or compared to a base level established in a control subject, or group of subjects.

5. A method according to claim 4 wherein the control subject is a male or a nonmenopausal female.

6. (canceled)

7. A method of treating and/or preventing cancer comprising applying a magnetic product to a human or animal body wherein the magnetic product comprises a magnet having positive and negative poles, the magnet having a metallic element which distorts the magnetic field distribution around the magnet so as to accentuate the magnetic field in the vicinity of the negative pole of the magnet.

8. A use or method according to claim 7 wherein the cancer is a cancer is a female specific cancer.

9. (canceled)

10. (canceled)

11. A method of increasing the level of IGFBP-3 in a subject comprising applying a magnetic product to the subject wherein the magnetic product comprises a magnet having positive and negative poles, the magnet having a metallic element which distorts the magnetic field distribution around the magnet so as to accentuate the magnetic field in the vicinity of the negative pole of the magnet.

12. (canceled)

13. (canceled)

14. The method according to claim 11 wherein the magnetic product is applied to the pelvic region of a female human.

15. (canceled)

16. The method according to any one of claims 1, 7 or 11 in which the magnetic product is applied to the body for 24 hours a day.

17. The method according to any one of claims 1, 7 or 11 wherein the magnetic product is applied to the body for from 7 to 28 days, 7 to 14 days or 7 to 21 days.

18. The method according to claim 17 in which the magnetic product is applied to the body for a continuous period of at least 10 days.

19. The method according to any one of claims 1, 7 or 11 wherein the magnetic product comprises a plurality of magnets.

20. The method according to claim 19 wherein the magnetic product comprises four magnets.

21. The method according to any one of claims 1, 7 or 11 wherein one or more of the magnets has a strength of from about 1,800 G to about 2,800 G.

22. The method according to any one of claims 1, 7 or 11 wherein one or more of the magnets comprises neodymium.

23. (canceled)

24. The method according to any one of claims 1, 7 or 11 in which the metallic element comprises a directional plate.
